(19) United States
(12) Patent Application Publication
(10) Pub. No.: US 2022/0152032 A1
(43) Pub. Date: May 19, 2022

(54) SEXUAL THERAPY FORMULATION AND METHOD OF TREATMENT

(71) Applicant: PHARMA AMERICA HOLDING INC., Miami, FL (US)

(72) Inventors: Bjorn Erik Hansen, Miami, FL (US); Roar Adelsten, Stockholm (SE); Robert Speth, Davie, FL (US)

(21) Appl. No.: 17/525,248

(22) Filed: Nov. 12, 2021

Related U.S. Application Data
(60) Provisional application No. 63/184,007, filed on May 4, 2021.
(60) Provisional application No. 63/113,615, filed on Nov. 13, 2020.

(51) Int. Cl. A61K 31/519; A61K 31/335

(52) U.S. Cl. CPC A61K 31/519; A61K 31/335

(57) ABSTRACT

A sexual therapy formulation for treatment of a patient suffering from erectile dysfunction as well as low or reduced libido. The formulation includes an erection enhancement component comprising one or more of sildenafil citrate, vardenafil hydrochloride, tadalafil, avanafil, and/or an alternate phosphodiesterase-5 inhibitor. The formulation also includes a libido stimulation component comprising one or more of dehydroepiandrosterone, testosterone, dihydrotestosterone, L-arginine, L-citrulline, Vitamin E, fenugreek, ginseng, maca, yohimbine, and/or 3,4-methylenedioxymethamphetamine including isomers and/or racemic mixtures thereof. A synergistic effect results from the erection enhancement component combined with the libido stimulation component allowing the patient to obtain and maintain an erection thus permitting the patient to engage in intimate sexual activity, as well providing the patient with the desire to engage in intimate sexual activity. A method of treatment of a patient includes administering to a patient at least one unit dosage of a sexual therapy formulation.

### CROSS-REFERENCE TO RELATED APPLICATIONS

This application claims the benefit of U.S. Provisional Patent Application Ser. No. 63/113,615 filed on Nov. 13, 2020, and U.S. Provisional Patent Application Ser. No. 63/184,007 filed on May 4, 2021, each of which are incorporated by reference herein in their entirety.

### FIELD OF THE INVENTION

The present invention relates generally to a sexual therapy formulation and a method of treatment of a patient suffering from a sexual disorder with a sexual therapy formulation.

### BACKGROUND OF THE INVENTION

It has been estimated that nearly 40% of the adult population suffer from some form of sexual dysfunction, also known as a sexual disorder. Erectile dysfunction (“ED”) is among the most prevalent forms of sexual disorders in men, and it includes the inability of a man to achieve or maintain an erection which is firm enough to engage in sexual activity involving penile penetration. Although it is normal for most men to experience difficulty obtaining or maintaining an erection on occasion, when this occurs more frequently and/or it disrupts a person's sex life, the person may be diagnosed with ED. Total erectile dysfunction is more severe and results in a men who are completely unable to obtain an erection. Erectile dysfunction is generally believed to be the most common form of sexual dysfunction in men, and is believed to affect about one third of adult men, with about one half of all men experiencing some form of erectile dysfunction from time to time.

It is generally accepted that the risk of ED increases with age, and that occurrences of total ED increase from about 5% in men at age 40 to about 15% in men at age 70. While age is acknowledged as a factor leading to occurrences of ED, numerous other factors affect a man's ability to obtain and/or maintain an erection, including both physical factors and psychological factors.

Among the various physical factors which could affect a man's ability to obtain and maintain an erection are alcohol use, illicit drug use, smoking, diabetes, high cholesterol, heart disease, blocked blood vessels, obesity, metabolic syndrome, certain medications, including common blood pressure medications, sleep disorders, scar tissue inside the penis, Parkinson's disease, and multiple sclerosis, among others.

As noted above, there are also psychological factors which could result in an inability to obtain or maintain an erection including, but not limited to, anxiety, stress, depression, and underlying issues in an intimate relationship, just to name a few.

Another type of sexual disorder common in both men and women is a low or reduced libido or sex drive, i.e., a loss of desire to engage in sexual activity at all. Although not as common as erectile dysfunction, men suffering from some form of low or reduced libido have been estimated to be about 20% to 25% of all adult men in the United States. Similar to ED, occurrences of low or reduced libido appear to increase as men and women grow older.

Also similar to erectile dysfunction, the cause of low libido in men can arise from a variety of factors both physical and psychological. Among the physical factors which may negatively impact a man's libido are low testosterone levels, impaired dopaminergic function in the brain, diabetes, obesity, high blood pressure, prescription and/or over the counter medicines, exercise, including too little and too much, as well as drug and/or alcohol use. It is noteworthy that about 4 out of 10 men reportedly have low testosterone levels after age 45. Testosterone supplements are commonly prescribed to men suffering from low testosterone levels.

The psychological factors that may reduce libido in men are similar to those which may cause varying degrees of erectile dysfunction including anxiety, stress, depression, and/or interpersonal relationship problems, just to name a few. Counseling can be effective in assisting men suffering from a low or reduced libido resulting solely from psychological factors.

It will be appreciated, however, that some men suffer from some degree of both erectile dysfunction and a low or reduced libido, and since they have similar causes, both physical and psychological, it is believed in many cases that men are not properly diagnosed and/or treated. As will also be appreciated, while treatment for ED may provide a man with the physical wherewithal to engage in sexual activity, a lack of sex drive may prevent him for doing so. As will further be appreciated, a man treated for a low libido may regain the desire for sexual activity, however, without proper treatment for erectile dysfunction, he may not be physically able to perform the desired activity.

Another common sexual disorder affecting primarily women, but observed in both men and women, is hypoactive sexual desire disorder (“HSDD”). Hypoactive sexual desire disorder is a sexual dysfunction marked by an absence of sexual desire that in turn causes distress in a sufferer's relationship with their partner. The symptoms of HSDD include a lack of or loss of motivation to participate in sexual activity due to decreased or absent spontaneous desire, lack of or loss of sexual desire in response to erotic cues or stimulation, and/or lack of or loss of an ability to maintain interest in sexual activity. To be clinically classified, the symptoms of HSDD must last at least 6 months and must be accompanied by distress in a sufferer's relationship with his or her sexual partner.

Hypoactive sexual desire disorder is the most prevalent type of sexual disorder observed in women. It has been reported that the prevalence of low sexual desire is about 30% in premenopausal women and over 50% in postmenopausal women. Although also occurring in men, the prevalence is reportedly much lower in men.

Hypoactive sexual desire disorder is gaining more recognition as a sexual disorder amenable to medical treatments. However, efforts to develop therapies and drugs to treat HSDD have had limited success. Currently, there are no drugs approved by the U.S. Food and Drug Administration (“FDA”) for treatment of HSDD in men, and only two drugs, flibanserin, available under the brand name ADDYI®, and brenmelanotide, sold under the brand name VYLEESI®, are approved by the FDA for treatment of HSDD in women, however, these drugs exhibit limited efficacy and adverse side-effects and contraindications have been reported.

Accordingly, there is an established need for a solution to the foregoing problems and shortcomings in the present state of the art related to the treatment of persons afflicted with a sexual disorder.

### SUMMARY OF THE INVENTION

The present invention is directed to a sexual therapy formulation and a method of treatment of a patient with a sexual therapy formulation.

In a first implementation of the invention, a sexual therapy formulation includes an amount of an erection enhancement component and an amount of a libido stimulation component, wherein the erectile enhancer may comprise sildenafil citrate, the active ingredient in VIAGRA®, and the libido enhancer can include 3,4-methylenedioxymethamphetamine, more commonly known as MDMA, and, a synergistic effect results from the erection enhancement component combined with the libido stimulation component in the present formulation allows the patient to obtain and maintain an erection thus permitting the patient to engage in intimate sexual activity as well providing the patient with the desire to engage in intimate sexual activity.

In a second aspect, the sexual therapy formulation can include: an erection enhancement component comprising one or more of sildenafil citrate, vardenafil hydrochloride, tadalafil, and avanafil; a libido stimulation component comprising one or more of dehydroepiandrosterone, testosterone, dihydrotestosterone, L-arginine, L-citrulline, Vitamin E, fenugreek, ginseng, maca, yohimbine, and 3,4-methylenedioxymethamphetamine; wherein a synergistic effect results from the erection enhancement component combined with the libido stimulation component thereby allowing the patient to obtain and maintain an erection thus permitting the patient to engage in intimate sexual activity as well providing the patient with the desire to engage in intimate sexual activity.

In another aspect, the sexual therapy formulation may have an erection enhancement component comprising an amount of sildenafil citrate.

In a further aspect, the sexual therapy formulation can include a unit dosage of the formulation comprising a therapeutically effective amount of an erection enhancement component comprising sildenafil citrate, and further, wherein the therapeutically effective amount of sildenafil citrate is about 20 milligrams to about 100 milligrams.

In one other aspect, the sexual therapy formulation may have a libido stimulation component comprising an amount of 3,4-methylenedioxymethamphetamine.

In yet another aspect, the sexual therapy formulation can include a unit dosage of the formulation comprising a therapeutically effective amount of a libido stimulation component comprising 3,4-methylenedioxymethamphetamine, wherein the therapeutically effective amount of 3,4-methylenedioxymethamphetamine is about 30 milligrams to about 125 milligrams.

In still one further aspect, the sexual therapy formulation may include: an erection enhancement component selected from the group consisting of sildenafil citrate, vardenafil hydrochloride, tadalafil, and avanafil; a libido stimulation component selected from the group consisting of dehydroepiandrosterone, testosterone, dihydrotestosterone, L-arginine, L-citrulline, Vitamin E, fenugreek, ginseng, maca, yohimbine, and 3,4-methylenedioxymethamphetamine; wherein a synergistic effect results from the erection enhancement component combined with the libido stimulation component thereby allowing the patient to obtain and maintain an erection thus permitting the patient to engage in intimate sexual activity as well providing the patient with the desire to engage in intimate sexual activity.

In yet one other aspect, the sexual therapy formulation can include an erection enhancement component consisting of an amount of sildenafil citrate.

In still another aspect, the sexual therapy formulation may have a unit dosage of the formulation comprising a therapeutically effective amount of an erection enhancement component consisting of sildenafil citrate, and further, wherein the therapeutically effective amount of sildenafil citrate is about 20 milligrams to about 100 milligrams.

In one alternate aspect, the sexual therapy formulation may have a unit dosage of the formulation for self-administration and/or home treatment comprising a therapeutically effective amount of an erection enhancement component consisting of sildenafil citrate, and further, wherein the therapeutically effective amount of sildenafil citrate is about 20 milligrams to about 100 milligrams.

In yet one further aspect, the sexual therapy formulation can include a libido stimulation component consisting of an amount of 3,4-methylenedioxymethamphetamine.

In still one other aspect, the sexual therapy formulation may have a unit dosage of the formulation comprising a therapeutically effective amount of a libido stimulation component consisting of 3,4-methylenedioxymethamphetamine, wherein the therapeutically effective amount of 3,4-methylenedioxymethamphetamine is about 30 milligrams to about 125 milligrams.

In another alternate aspect, the sexual therapy formulation may have a unit dosage of the formulation for self-administration and/or home treatment comprising a therapeutically effective amount of a libido stimulation component consisting of 3,4-methylenedioxymethamphetamine, wherein the therapeutically effective amount of 3,4-methylenedioxymethamphetamine is about 30 milligrams to about 50 milligrams

These and other objects, features, and advantages of the present invention will become more readily apparent from the detailed description of the embodiments, which follow.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The terminology used herein is for the purpose of describing particular embodiments only and is not intended to be limiting of the invention. As used herein, the indefinite articles “a”, “an” and “the” should be understood to include plural reference unless the context clearly indicates otherwise.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either.” “one of,” “only one of,” or “exactly one of.”

As used herein, the terms “including”, “includes”, “having”, “has”, “with”, or variants thereof, are intended to be inclusive similar to the term “comprising.”

As used herein, the term “patient” refers to any male of female human being who is the recipient of a subject treatment. The terms “subject” and “patient” may be used interchangeably herein in reference to a patient.

As used herein, the term “component” or “components” refer to an elemental or composite molecule of any of a variety of therapeutic chemicals which may be naturally occurring in certain plants and/or animals and/or derived therefrom, or which may be wholly manufactured molecules which have no naturally occurring counterparts, and include, but are in no manner limited to, biologics and pharmaceutical compositions, and which may be incorporated into a sexual therapy formulation in accordance with the present invention.

As used herein, the term “formulation” refers to any combination of two or more components, as defined hereinabove, intended to elicit a desired physiological and/or psychological response from a patient to which the present sexual therapy formulation is administered.

A formulation in accordance with the present invention may also include or be packaged with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, nanomaterials, drug conjugates intended to improve drug distribution to specific tissues of the body, and the like. Specifically, the pharmaceutically acceptable carriers, excipients, binders, and fillers contemplated for use in the practice of the present invention are those which render the compounds of the invention amenable for delivery to a patient by any of a variety of delivery mechanisms including, but not limited to, orally, intravenous, transdermal, topical, rectally, inhalation, and the like. Moreover, the packaging material may be biologically inert or lack bioactivity, such as plastic polymers, silicone, etc., which may be processed internally by the subject without affecting the efficacy of the formulation packaged and/or delivered therewith.

The term “therapeutically effective amount” as used with reference to the present sexual therapy formulation and/or components thereof as described herein refers to the quantity of the sexual therapy formulation and/or components thereof necessary to render the desired therapeutic result. For example, an effective amount is a level effective to treat, cure, or alleviate the symptoms of a disorder for which the therapeutic formulation is being administered. Amounts effective for the particular therapeutic goal sought will depend upon a variety of factors including: the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage will necessarily occur depending upon the condition and characteristics of the subject being treated, and the physician or other individual administering treatment will, in any event, determine the appropriate dosage for each individual patient.

As used herein, the term “administering” refers to providing a therapeutically effective amount of the present sexual therapy formulation and/or components thereof to a patient via any of a number of potential delivery mechanisms including, but not limited to, oral, intravenous, transdermal, topical, rectally and/or inhalation, and the like. The formulation and/or components thereof of the present invention can be administered individually, but may also be administered with other compounds, excipients, fillers, binders, carriers or other vehicles selected based upon the chosen route of administration and standard pharmaceutical practice. Administration may be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; synthetic polymers; microspheres; nanoparticles; and the like.

As used herein, “disorder” refers to a disorder, disease or condition, or other departure from healthy or normal biological activity, and the terms can be used interchangeably. The terms would refer to any condition that impairs healthy sexual function including, but not limited to, one or more of erectile dysfunction (“ED”), low libido, hypoactive sexual desire disorder (“HSDD”), dysfunctional genital response to sexual stimulation in both men and women, female sexual arousal disorder, female sexual interest/arousal disorder, female orgasmic disorder, genito-pelvic pain/penetration disorder, among others. The disorder or condition may be caused by any of a number of physical and/or psychological factors. The disorder or condition may be caused by sporadic or heritable genetic abnormalities. The disorder or condition may also be caused by non-genetic abnormalities. The disorder or condition may also be caused by injuries to a subject from environmental factors.

As used herein, “treatment” or “treating” refers to arresting or inhibiting, or attempting to arrest or inhibit, the development or progression of a disorder and/or causing, or attempting to cause, the reduction, suppression, regression, or remission of a disorder and/or a symptom thereof. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms.

As before, the present invention is directed to a sexual therapy formulation and a method of treatment of a patient with a sexual therapy formulation. More in particular, in one embodiment, the present invention is directed to a sexual therapy formulation and a method of treatment of men who experience at least some degree of erectile dysfunction, among the most common sexual disorder in men, and who also experience a low or reduced libido or sex drive, with a sexual therapy formulation. In at least one other embodiment, the present invention is directed to a sexual therapy formulation and a method of treatment of women who experience at least some degree of sexual dysfunction.

In one embodiment, a sexual therapy formulation in accordance with the present invention comprises an erection enhancement component and a libido stimulation component. In at least one further embodiment, a sexual therapy formulation comprises a therapeutically effective amount of an erection enhancement component and a therapeutically effective amount of a libido stimulation component, wherein the combination of the erection enhancement component and the libido stimulation component produce synergistically beneficial results in a patient to which the formulation is administered. The present invention further comprises a suggested dosage regimen for the treatment of a patient with the present sexual therapy formulation.

A number of compositions have been developed in recent years to aid men who experience at least some degree of erectile dysfunction from time to time, i.e., men who experience difficulty having and maintaining an erection, also referred to as impotence. While impotence may be experienced by men of any age, it is more commonly an issue which arises as men become older. Of course, a number of other factors are known to have an impact on the frequency and/or severity of erectile dysfunction in men including lifestyle, exercise, weight, smoking, drug and/or alcohol use, etc., as discussed hereinabove.

Among the compositions which have been developed for the treatment of erectile dysfunction in men are sildenafil citrate, which is the active ingredient in the well-known erectile dysfunction medication VIAGRA®, vardenafil hydrochloride, also known as LEVITRA® or STAXYN®, tadalafil, the active ingredient in CIALIS®, and avanafil, also known as STENDRA®. In at least one embodiment, an alternate phosphodiesterase-5 (“PDE5”) inhibitor may be administered as an erection enhancement component in accordance with the present invention.

While sildenafil citrate and other phosphodiesterase-5 inhibitors are not presently approved for use in women, there have been some reports of efficacy in women, for example, women who have a dysfunctional genital response to sexual stimulation. Thus, women may also benefit from the present sexual therapy formulation.

Each of the foregoing compositions, as well as generic equivalents thereof, are oral medications that essentially enhance the effects of nitric oxide, a natural chemical produced in the body by both men and women, which relaxes vascular smooth muscles in a man's penis and in the genitalia, thereby increasing blood flow to the genitalia of both men and women. The result is an increase in blood flow to the penis which allows most men to obtain and maintain an erection and allows many women to experience increased vaginal secretions in response to sexual stimulation. Unfortunately, many men who suffer from erectile dysfunction and women who suffer from vaginal dryness also suffer from a low or reduced libido, as noted above.

A number of factors are known to affect the libido in men and women including, but in no manner limited to: biological factors, such as testosterone and estrogen levels; psychological factors, such as stress levels and/or insecurity due to prior instances of erectile dysfunction; and/or social factors, including intimate relationships. Also as before, a number of compositions have been put forth to aid men and women experiencing a low or reduced libido or sex drive, including, but not limited to, dehydroepiandrosterone, or DHEA, a hormone produced by the adrenal glands which the body then uses to produce sex hormones, testosterone, dihydrotestosterone, L-arginine and/or L-citrulline, amino acids associated with nitric oxide formation, vitamin E, a composition of eight fat soluble compounds that include four tocopherols and four tocotrienols, and which is known, among other things, to improve sperm motility and form, A number of herbal supplements have also been suggested to aid men and women experiencing low libido including, but not limited to, fenugreek, ginseng, maca, and yohimbine.

Further, 3,4-methylenedioxymethamphetamine, more commonly known as MDMA, or in the recreational drug culture as Ecstasy or Molly, is believed to increase sexual desire in both men and women, as well as to intensify the orgasmic experience in both. However, 3,4-methylenedioxymethamphetamine or MDMA is also known to impair the ability of some men to obtain and/or maintain an erection. Administration of MDMA to a patient, even under a clinical trial setting, is currently subject to regulation by various federal and state governmental agencies.

In view of the foregoing, in at least one embodiment, the present sexual therapy formulation has been developed to provide a treatment for men afflicted with some level of erectile dysfunction combined with a low or reduced libido or sex drive. As before, in accordance with at least one embodiment of the present invention, a sexual therapy formulation comprises an erectile enhancement component combined with a libido stimulation component.

As such, in one embodiment of the present invention, a sexual therapy formulation includes an erection enhancement component comprising one or more of sildenafil citrate, vardenafil hydrochloride, tadalafil, avanafil, and/or an alternate phosphodiesterase-5 inhibitor, and a libido stimulation component comprising one or more of dehydroepiandrosterone, testosterone, dihydrotestosterone, L-arginine, L-citrulline, Vitamin E, fenugreek, ginseng, maca, yohimbine, 3,4-methylenedioxymethamphetamine, and/or R(−) or S(+) isomers and/or racemic mixtures of 3,4-methylenedioxymethamphetamine.

In one further embodiment, a sexual therapy formulation in accordance with the present invention includes an erection enhancement component comprising an amount of sildenafil citrate. More in particular, a unit dosage of a sexual therapy formulation in accordance with at least one embodiment of the present invention includes a therapeutically effective amount of an erection enhancement component comprising sildenafil citrate.

In yet another embodiment, a unit dosage of the present formulation includes a therapeutically effective amount of sildenafil citrate of about 20 milligrams to about 100 milligrams. In still one further embodiment, a unit dosage of a sexual therapy formulation comprises a therapeutically effective amount of sildenafil citrate of about 20 milligrams, or about 50 milligrams, or about 100 milligrams. As will be appreciated, the therapeutically effective amount of sildenafil citrate incorporated into a unit dosage of the present formulation may vary for administration to patients experiencing varying degrees of erectile dysfunction, which may be characterized as, by way of example only, mild, moderate, or severe.

As one example, a unit dosage of a sexual therapy formulation in accordance with the present invention for a patient experiencing mild symptoms of erectile dysfunction may comprise a therapeutically effective amount of sildenafil citrate of about 20 milligrams. By way of comparison, the maximum recommended dosage of sildenafil citrate for patient's experiencing any degree of hepatic impairment is 25 milligrams. As one further example, a unit dosage of a sexual therapy formulation in accordance with the present invention for a patient experiencing moderate symptoms of erectile dysfunction comprises a therapeutically effective amount of sildenafil citrate of about 50 milligrams, and in yet another embodiment, a unit dosage of a sexual therapy formulation for a patient experiencing severe symptoms of erectile dysfunction may comprise a therapeutically effective amount of sildenafil citrate of about 100 milligrams.

In one alternate embodiment, a unit dosage of a sexual therapy formulation may be developed specifically for self-administration and/or home treatment and may comprise a therapeutically effective amount of an erection enhancement component consisting of sildenafil citrate, and further, wherein the therapeutically effective amount of sildenafil citrate is about 20 milligrams to about 100 milligrams.

In one further embodiment of the present invention, a sexual therapy formulation includes an erection enhancement component selected from the group consisting of sildenafil citrate, vardenafil hydrochloride, tadalafil, avanafil, and an alternate phosphodiesterase-5 (“PDE5”) inhibitor, and a libido stimulation component comprising one or more of dehydroepiandrosterone, testosterone, dihydrotestosterone, L-arginine, L-citrulline, Vitamin E, fenugreek, ginseng, maca, yohimbine, 3,4-methylenedioxymethamphetamine, an R(−) isomer of 3,4-methylenedioxymethamphetamine, an S(+) isomer of 3,4-methylenedioxymethamphetamine, and a racemic mixture of 3,4-methylenedioxymethamphetamine.

More in particular, a sexual therapy formulation in accordance with at least one embodiment of the present invention includes an erection enhancement component consisting of an amount of sildenafil citrate. Furthermore, a unit dosage of a sexual therapy formulation in accordance with at least one embodiment of the present invention includes a therapeutically effective amount of an erection enhancement component consisting of sildenafil citrate.

In yet one further embodiment, a unit dosage of the present formulation includes a therapeutically effective amount of sildenafil citrate of about 20 milligrams to about 100 milligrams, and in still one other embodiment, a unit dosage of a sexual therapy formulation comprises a therapeutically effective amount of sildenafil citrate of about 20 milligrams, or about 50 milligrams, or about 100 milligrams. As will be appreciated, and as before, the therapeutically effective amount of sildenafil citrate incorporated into a unit dosage of the present formulation may vary for administration to patients experiencing varying degrees of erectile dysfunction, which may be characterized as, by way of example only, mild, moderate, or severe.

Once again, as one example, a unit dosage of a sexual therapy formulation in accordance with the present invention for a patient experiencing mild symptoms of erectile dysfunction may comprise a therapeutically effective amount of sildenafil citrate of about 20 milligrams. As before, the maximum recommended dosage of sildenafil citrate for patient's experiencing any degree of hepatic impairment is 25 milligrams. As another example, a unit dosage of a sexual therapy formulation in accordance with the present invention for a patient experiencing moderate symptoms of erectile dysfunction comprises a therapeutically effective amount of sildenafil citrate of about 50 milligrams, and in yet one further example, a unit dosage of a sexual therapy formulation for a patient experiencing severe symptoms of erectile dysfunction may comprise a therapeutically effective amount of sildenafil citrate of about 100 milligrams.

A sexual therapy formulation in accordance with the present invention also comprises a libido stimulation component, as disclosed hereinabove. In accordance with one embodiment, a sexual therapy formulation comprises a libido stimulation component comprising one or more of dehydroepiandrosterone, testosterone, dihydrotestosterone, L-arginine, L-citrulline, Vitamin E, fenugreek, ginseng, maca, yohimbine, 3,4-methylenedioxymethamphetamine, and/or R(−) or S(+) isomers and/or racemic mixtures of 3,4-methylenedioxymethamphetamine. A synergistic effect results from the erection enhancement component combined with the libido stimulation component in the present formulation which allows a male patient to obtain and maintain an erection and which allows a female patient to have an appropriate physiological response to sexual stimulation, thus permitting the patient to engage in intimate sexual activity as well providing the patient with the desire to engage in intimate sexual activity. As used herein, “intimate sexual activity” shall mean sexual activity which includes, but is not limited to, penile penetration of a sexual partner performed by or to the patient.

In at least one embodiment of the present invention, a sexual therapy formulation includes a libido stimulation component comprising an amount of 3,4-methylenedioxymethamphetamine and/or R(−) or S(+) isomers and/or racemic mixtures thereof. More in particular, a unit dosage of a sexual therapy formulation in accordance with at least one embodiment of the present invention includes a therapeutically effective amount of a libido stimulation component comprising 3,4-methylenedioxymethamphetamine and/or R(−) or S(+) isomers and/or racemic mixtures thereof.

More in particular, 3,4-methylenedioxymethamphetamine has a stereo isometric center which divides it into two stereoisomers, namely, an R(−) isomer and an S(+) isomer. Of these two isomers, the R(−) isomer is more potent than the S(+) isomer at stimulating serotonin release, although both are reportedly equipotent at increasing oxytocin release. The advantage of resolving the R(−)isomer of 3,4-methylenedioxymethamphetamine for therapeutic usage is the potential to use a lesser or an equivalent amount of the R(−) isomer of 3,4-methylenedioxymethamphetamine compared with racemic mixtures of 3,4-methylenedioxymethamphetamine to get an equivalent therapeutic effect, with a substantial reduction in potential toxicity. Further, it has been reported that the R(−) isomer of 3,4-methylenedioxymethamphetamine has less abuse potential, less hyperthermic effects, less neurotoxicity, less hypertensive effects than the S(+) isomer or racemic mixtures of 3,4-methylenedioxymethamphetamine, with equivalent prosocial effects.

In one embodiment, a unit dosage of the present formulation includes a therapeutically effective amount of 3,4-methylenedioxymethamphetamine, and/or R(−) or S(+) isomers and/or racemic mixtures thereof, of about 30 milligrams to about 125 milligrams. In one further embodiment, a therapeutically effective amount of 3,4-methylenedioxymethamphetamine, and/or R(−) or S(+) isomers and/or racemic mixtures thereof, in a unit dosage of a sexual therapy formulation in accordance with the present invention is about 30 milligrams, or about 40 milligrams, or about 50 milligrams, or about 60 milligrams, or about 70 milligrams, or about 80 milligrams, or about 90 milligrams, or about 100 milligrams, or about 110 milligrams, or about 125 milligrams, which is the maximum recommended unit dosage of 3,4-methylenedioxymethamphetamine to avoid potential neurotoxicity. A therapeutically effective amount of 3,4-methylenedioxymethamphetamine, and/or R(−) or S(+) isomers and/or racemic mixtures thereof, incorporated into a unit dosage of the present sexual therapy formulation can vary based upon a degrees of low or reduced libido a patient is experiencing, which may be characterized, by way of example only, along a sliding scale ranging from mild, to moderate, to severe.

Further factors to be considered in determining a therapeutically effective amount of 3,4-methylenedioxymethamphetamine are whether it is being administered to a patient for the first time, other medications the patient may be taking, as well as a patient's weight. Specifically, in accordance with at least one embodiment of the present invention, a therapeutically effective amount of 3,4-methylenedioxymethamphetamine is calculated based on a patient's weight in a ratio of 1.5 milligrams of 3,4-methylenedioxymethamphetamine per ten (10) kilograms of patient body weight. As before, a maximum recommend unit dosage of 3,4-methylenedioxymethamphetamine is 125 milligrams, regardless of the patient's body weight, so as to avoid neurotoxicity. Higher dosages of the R(−) isomer of 3,4-methylenedioxymethamphetamine may be possible due to its reduced neurotoxicity profile relative to 3,4-methylenedioxymethamphetamine and the S(+) isomer and racemic mixtures thereof.

As before, in one alternate embodiment, a unit dosage of a sexual therapy formulation may be developed specifically for self-administration and/or home treatment and may comprise a therapeutically effective amount of a libido stimulation component consisting of 3,4-methylenedioxymethamphetamine, and/or R(−) or S(+) isomers and/or racemic mixtures thereof, and further, wherein the therapeutically effective amount of 3,4-methylenedioxymethamphetamine, and/or R(−) or S(+) isomers and/or racemic mixtures thereof, is about 30 milligrams to about 50 milligrams.

In yet one further alternate embodiment, a sexual therapy formulation comprises a libido stimulation component selected from the group consisting of dehydroepiandrosterone, testosterone, dihydrotestosterone, L-arginine, L-citrulline, Vitamin E, fenugreek, ginseng, maca, yohimbine, 3,4-methylenedioxymethamphetamine, R(−) or S(+) isomers of 3,4-methylenedioxymethamphetamine and/or racemic mixtures of 3,4-methylenedioxymethamphetamine. As before, a synergistic effect results from the erection enhancement component combined with the libido stimulation component in the present formulation allowing a patient to obtain and maintain an erection and/or experience an appropriate physiological response to sexual stimulation, thus permitting the patient to engage in intimate sexual activity as well providing the patient with the desire to engage in intimate sexual activity.

In at least one embodiment, a sexual therapy formulation includes a libido stimulation component consisting of an amount of 3,4-methylenedioxymethamphetamine, an R(−) isomer of 3,4-methylenedioxymethamphetamine, an S(+) isomer of 3,4-methylenedioxymethamphetamine and/or a racemic mixtures of 3,4-methylenedioxymethamphetamine. More in particular, a unit dosage of a sexual therapy formulation in accordance with at least one embodiment of the present invention consists of a therapeutically effective amount of a libido stimulation component consisting of 3,4-methylenedioxymethamphetamine, an R(−) isomer of 3,4-methylenedioxymethamphetamine, an S(+) isomer of 3,4-methylenedioxymethamphetamine and/or a racemic mixtures of 3,4-methylenedioxymethamphetamine.

In one embodiment, a unit dosage of the present formulation includes a therapeutically effective amount of 3,4-methylenedioxymethamphetamine, and/or R(−) or S(+) isomers and/or racemic mixtures thereof, of about 30 milligrams to about 125 milligrams. In one further embodiment, a therapeutically effective amount of 3,4-methylenedioxymethamphetamine, and/or R(−) or S(+) isomers and/or racemic mixtures thereof, in a unit dosage of a sexual therapy formulation in accordance with the present invention is about 30 milligrams, or about 40 milligrams, or about 50 milligrams, or about 60 milligrams, or about 70 milligrams, or about 80 milligrams, or about 90 milligrams, or about 100 milligrams, or about 110 milligrams, or about 125 milligrams, which, as before, is the maximum recommended unit dosage of 3,4-methylenedioxymethamphetamine to avoid potential neurotoxicity. As before, the therapeutically effective amount of 3,4-methylenedioxymethamphetamine, and/or R(−) or S(+) isomers and/or racemic mixtures thereof, incorporated into a unit dosage of the present sexual therapy formulation can vary based upon a degrees of low or reduced libido a patient is experiencing, which may be characterized, by way of example only, along a sliding scale ranging from mild, to moderate, to severe.

Additional factors which are considered in order to determine therapeutically effective amount of 3,4-methylenedioxymethamphetamine, and/or R(−) or S(+) isomers and/or racemic mixtures thereof once again, are whether it is being administered for the first time to a patient, other medications the patient may be taking, as well as a patient's weight. Specifically, in accordance with at least one embodiment of the present invention, a therapeutically effective amount of 3,4-methylenedioxymethamphetamine is calculated based on a patient's weight in a ratio of 1.5 milligrams of 3,4-methylenedioxymethamphetamine per ten (10) kilograms of patient body weight. Once again, a maximum recommend unit dosage of 3,4-methylenedioxymethamphetamine is 125 milligrams, regardless of the patient's body weight, so as to avoid neurotoxicity, however, as before, higher dosages of the R(−) isomer of 3,4-methylenedioxymethamphetamine may be possible due to its reduced neurotoxicity profile relative to 3,4-methylenedioxymethamphetamine and the S(+) isomer thereof.

The following tables are illustrative of various combinations of therapeutically effective amounts of an erectile enhancement component and therapeutically effective amounts of a libido stimulation component in a unit dosage of a sexual therapy formulation, in accordance with at least one embodiment of the present invention. More in particular, Table 1 is illustrative of various combinations of a therapeutically effective amount of an erectile enhancement component comprising sildenafil citrate and a therapeutically effective amount of a libido stimulation component comprising 3,4-methylenedioxymethamphetamine for a patient experiencing a mild degree of erectile dysfunction; Table 2 is illustrative of various combinations of a therapeutically effective amount of an erectile enhancement component comprising sildenafil citrate and a therapeutically effective amount of a libido stimulation component comprising 3,4-methylenedioxymethamphetamine for a patient experiencing a moderate degree of erectile dysfunction; and, Table 3 is illustrative of various combinations of a therapeutically effective amount of an erectile enhancement component comprising sildenafil citrate and a therapeutically effective amount of a libido stimulation component comprising 3,4-methylenedioxymethamphetamine for a patient experiencing a severe degree of erectile dysfunction.

TABLE 1
Sexual Therapy Formulation - Mild ED

| Amount of | Amount of | |
|---|---|---|
| sildenafil citrate | 3,4-methylenedioxymethamphetamine | |
| 20 milligrams | 30 | milligrams |
| 20 milligrams | 40 | milligrams |
| 20 milligrams | 50 | milligrams |
| 20 milligrams | 60 | milligrams |
| 20 milligrams | 70 | milligrams |
| 20 milligrams | 80 | milligrams |
| 20 milligrams | 90 | milligrams |
| 20 milligrams | 100 | milligrams |
| 20 milligrams | 110 | milligrams |
| 20 milligrams | 125 | milligrams |

TABLE 2
Sexual Therapy Formulation - Moderate ED

| Amount of | Amount of | |
|---|---|---|
| sildenafil citrate | 3,4-methylenedioxymethamphetamine | |
| 50 milligrams | 30 | milligrams |
| 50 milligrams | 40 | milligrams |
| 50 milligrams | 50 | milligrams |
| 50 milligrams | 60 | milligrams |
| 50 milligrams | 70 | milligrams |
| 50 milligrams | 80 | milligrams |
| 50 milligrams | 90 | milligrams |
| 50 milligrams | 100 | milligrams |
| 50 milligrams | 110 | milligrams |
| 50 milligrams | 125 | milligrams |

TABLE 3
Sexual Therapy Formulation - Severe ED

| Amount of | Amount of | |
|---|---|---|
| sildenafil citrate | 3,4-methylenedioxymethamphetamine | |
| 100 milligrams | 30 | milligrams |
| 100 milligrams | 40 | milligrams |
| 100 milligrams | 50 | milligrams |
| 100 milligrams | 60 | milligrams |
| 100 milligrams | 70 | milligrams |
| 100 milligrams | 80 | milligrams |
| 100 milligrams | 90 | milligrams |
| 100 milligrams | 100 | milligrams |
| 100 milligrams | 110 | milligrams |
| 100 milligrams | 125 | milligrams |

As noted above, the combination of an erection enhancement component and a libido stimulation component in the present sexual therapy formulation produces synergistically beneficial results in a patient to which the formulation is administered. As one example, sildenafil citrate lowers blood pressure, which serves to counteract the propensity of 3,4-methylenedioxymethamphetamine to increase blood pressure. In addition, sildenafil citrate can reduce the neurotoxicity of 1,4-methylenedioxymethamphetamine by virtue of its anti-inflamatory and antioxidant actions. Thus, a formulation having sildenafil citrate in combination with 3,4-methylenedioxymethamphetamine for sexual enhancement should reduce the risk of neurotoxicity and/or cardiovascular dysfunction.

As before, the present invention further comprises a method of treatment of a patient with a sexual therapy formulation. In at least one embodiment the method includes conducting an initial evaluation of a patient including a review of the patient's physical, medical and sexual history by a licensed physician and/or a licensed sexual therapist. In one embodiment, conducting an initial evaluation can include or incorporate an interview with the patient's sexual partner, in instances wherein the patient is involved in a relationship with a specific sexual partner.

A method of treatment of a patient with a sexual therapy formulation in accordance with the present invention in at least one embodiment further includes developing an initial treatment regimen for the patient. An initial treatment regimen in accordance with the present invention includes prescribing a specific unit dosage of a sexual therapy formulation for administration to the patient upon implementing an initial sexual therapy session, wherein the unit dosage is determined based upon the various factors delineated hereinabove. The initial treatment regimen will also specify any physical constraints or conditions to be placed upon the patient during an initial sexual therapy session. As before, a licensed physician and/or a licensed sexual therapist will develop an initial treatment regimen in accordance with at least one embodiment of the present invention.

In at least one embodiment, a method of treatment of a patient with a sexual therapy formulation in accordance with the present invention further comprises administering at least one unit dosage of a sexual therapy formulation to a patient. In at least one further embodiment, the present method includes administering at least one unit dosage of a sexual therapy formulation to a patient in a controlled environment with a designated sexual partner present. In yet another alternate embodiment, the present method includes prescribing a unit dosage of a sexual therapy formulation developed specifically for self-administration and/or home treatment.

A controlled environment in accordance with the present invention can include a clinical environment such as a private room in a physician's office or a therapist's office which is furnished to facilitate intimate sexual activity between a patient and a designated sexual partner. In accordance with at least one embodiment of the present invention, a controlled environment may comprise a dedicated sexual therapy facility wherein a patient and a designated sexual partner can spend an entire weekend or longer in a resort like atmosphere conducive to intimate sexual relations. In still one further embodiment, a dedicated sexual therapy facility can include all of the amenities associated with a destination vacation getaway, wherein patients and their designated sexual partners are able to enjoy the facility and grounds and all of the amenities thereof with other patients and their partners. Further, each patient and their designated sexual partner can also avail themselves of a private dedicated room or rooms in which they may engage in intimate sexual relations during sexual therapy sessions, all under the guidance, once again, of licensed physicians and/or licensed sexual therapists.

The present method of treatment of a patient with a sexual therapy formulation in accordance with the present invention further comprises implementing a sexual therapy session within a predetermined timeframe from a time of administration of a sexual therapy formulation to a patient. During each sexual therapy session, a patient is encouraged to engage in intimate sexual activity, as defined hereinabove, with a designated sexual partner. Depending on the physiology of the patient and the unit dosage of the sexual therapy formulation specified for the patient in accordance with the present invention, the predetermined timeframe may be as little as 30 to 40 minutes or it may be up to several hours after administration of the sexual therapy formulation to the patient.

In accordance with the present invention, implementing a sexual therapy session may occur in total privacy between a patient and a patient's designated sexual partner. In at least one embodiment, a sexual therapy session occurs in privacy between the patient and the patient's designated sexual partner, however, the sessions are recorded for review with the patient, and in some instances, with the patient's designated sexual partner, during a post therapy session evaluation. In still another embodiment of the present method, a sexual therapy session between the patient and the patient's designated sexual partner occurs in the presence of a licensed physician and/or a licensed sexual therapist.

A method of treatment of a patient with a sexual therapy formulation in accordance with the present invention further comprises a post therapy session evaluation. More in particular, a post therapy session evaluation in accordance with at least one embodiment of the present invention comprises an assessment of the patient's physical sexual performance, as well as the patient's psychological impressions while engaging in sexual activity during a sexual therapy session. As before, the assessment will be conducted by a licensed physician and/or a licensed sexual therapist and will be based upon the patient's recounting of a totally private sexual therapy session and/or a review of a video recording and/or the licensed physician and/or licensed therapist notes taking during a real time observation of the patient during a sexual therapy session.

A method of treatment of a patient with a sexual therapy formation in accordance with the present invention, in at least one embodiment, further comprises developing a subsequent treatment regimen for the patient. In at least one embodiment, a subsequent treatment regimen is developed after each post therapy session evaluation. Similar to an initial treatment regimen in accordance with the present invention, a subsequent treatment regimen includes prescribing a specific unit dosage of a sexual therapy formulation for administration to the patient upon implementing a subsequent sexual therapy session, wherein the unit dosage is determined based upon the various factor delineated hereinabove, as well as the patient's sexual performance during one or more preceding sexual therapy sessions. Each subsequent treatment regimen will also specify any physical constraints or conditions to be placed upon the patient during each subsequent sexual therapy session. As before, a licensed physician or a licensed sexual therapist will develop each subsequent treatment regimen in accordance with at least one embodiment of the present invention.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

What is claimed is:

1. A sexual therapy formulation for treatment of a patient suffering from some degree of erectile dysfunction as well as low or reduced libido, said formulation comprising:
an erection enhancement component comprising one or more of sildenafil citrate, vardenafil hydrochloride, tadalafil, and avanafil;
a libido stimulation component comprising one or more of dehydroepiandrosterone, testosterone, dihydrotestosterone, L-arginine, L-citrulline, Vitamin E, fenugreek, ginseng, maca, yohimbine, 3,4-methylenedioxymethamphetamine, an R(−) isomer of 3,4-methylenedioxymethamphetamine, an S(+) isomer of 3,4-methylenedioxymethamphetamine, and a racemic mixture of 3,4-methylenedioxymethamphetamine; and
a synergistic effect produced from said erection enhancement component combined with said libido stimulation component allows the patient to obtain and maintain an erection thus permitting the patient to engage in intimate sexual activity as well providing the patient with the desire to engage in intimate sexual activity.

2. The sexual therapy formulation as recited in claim 1, wherein said erection enhancement component comprises an amount of sildenafil citrate.

3. The sexual therapy formulation as recited in claim 2, wherein a unit dosage of said formulation comprises a therapeutically effective amount of said erection enhancement component comprising sildenafil citrate.

4. The sexual therapy formulation as recited in claim 3, wherein said therapeutically effective amount of sildenafil citrate is about 20 milligrams to about 100 milligrams.

5. The sexual therapy formulation as recited in claim 1, wherein said libido stimulation component comprises an amount of 3,4-methylenedioxymethamphetamine, said R(−) isomer of 3,4-methylenedioxymethamphetamine, said S(−) isomer of 3,4-methylenedioxymethamphetamine, and/or said racemic mixture of 3,4-methylenedioxymethamphetamine.

6. The sexual therapy formulation as recited in claim 5, wherein a unit dosage of said formulation comprises a therapeutically effective amount of said libido stimulation component comprising 3,4-methylenedioxymethamphetamine, said R(−) isomer of 3,4-methylenedioxymethamphetamine, said S(+) isomer of 3,4-methylenedioxymethamphetamine, and/or said racemic mixture of 3,4-methylenedioxymethamphetamine.

7. The sexual therapy formulation as recited in claim 6, wherein said therapeutically effective amount of 3,4-methylenedioxymethamphetamine, said R(−) isomer of 3,4-methylenedioxymethamphetamine, said S(+) isomer of 3,4-methylenedioxymethamphetamine, and/or said racemic mixture of 3,4-methylenedioxymethamphetamine, is about 30 milligrams to about 125 milligrams.

8. The sexual therapy formulation as recited in claim 4, wherein said libido stimulation component comprises an amount of 3,4-methylenedioxymethamphetamine, said R(−) isomer of 3,4-methylenedioxymethamphetamine, said S(+) isomer of 3,4-methylenedioxymethamphetamine, and/or said racemic mixture of 3,4-methylenedioxymethamphetamine.

9. The sexual therapy formulation as recited in claim 8, wherein a unit dosage of said formulation comprises a therapeutically effective amount of said libido stimulation component comprising 3,4-methylenedioxymethamphetamine, said R(−) isomer of 3,4-methylenedioxymethamphetamine, said S(+) isomer of 3,4-methylenedioxymethamphetamine, and/or said racemic mixture of 3,4-methylenedioxymethamphetamine.

10. The sexual therapy formulation as recited in claim 9, wherein said therapeutically effective amount of 3,4-methylenedioxymethamphetamine, said R(−) isomer of 3,4-methylenedioxymethamphetamine, said S(+) isomer of 3,4-methylenedioxymethamphetamine, and/or said racemic mixture of 3,4-methylenedioxymethamphetamine, is about 30 milligrams to about 125 milligrams.

11. A sexual therapy formulation for treatment of a patient suffering from some degree of erectile dysfunction as well as low or reduced libido, said formulation comprising:
an erection enhancement component selected from the group consisting of sildenafil citrate, vardenafil hydrochloride, tadalafil, and avanafil;
a libido stimulation component selected from the group consisting of dehydroepiandrosterone, testosterone, dihydrotestosterone, L-arginine, L-citruline, Vitamin E, fenugreek, ginseng, maca, yohimbine, 3,4-methylenedioxymethamphetamine, an R(−) isomer of 3,4-methylenedioxymethamphetamine, an S(+) isomer of 3,4-methylenedioxymethamphetamine, and a racemic mixture of 3,4-methylenedioxymethamphetamine; and
a synergistic effect produced from said erection enhancement component combined with said libido stimulation component allows the patient to obtain and maintain an erection thus permitting the patient to engage in intimate sexual activity as well providing the patient with the desire to engage in intimate sexual activity.

12. The sexual therapy formulation as recited in claim 11, wherein said erection enhancement component consists of an amount of sildenafil citrate.

13. The sexual therapy formulation as recited in claim 12, wherein a unit dosage of said formulation comprises a therapeutically effective amount of said erection enhancement component consisting of sildenafil citrate.

14. The sexual therapy formulation as recited in claim 13, wherein said therapeutically effective amount of sildenafil citrate is about 20 milligrams to about 100 milligrams.

15. The sexual therapy formulation as recited in claim 11, wherein said libido stimulation component consists of an amount of 3,4-methylenedioxymethamphetamine, said R(−) isomer of 3,4-methylenedioxymethamphetamine, said S(+) isomer of 3,4-methylenedioxymethamphetamine, or said racemic mixture of 3,4-methylenedioxymethamphetamine.

16. The sexual therapy formulation as recited in claim 15, wherein a unit dosage of said formulation comprises a therapeutically effective amount of said libido stimulation component consisting of 3,4-methylenedioxymethamphetamine, said R(−) isomer of 3,4-methylenedioxymethamphetamine, said S(+) isomer of 3,4-methylenedioxymethamphetamine, or said racemic mixture of 3,4-methylenedioxymethamphetamine.

17. The sexual therapy formulation as recited in claim 16, wherein said therapeutically effective amount of 3,4-methylenedioxymethamphetamine, said R(−) isomer of 3,4-methylenedioxymethamphetamine, said S(−) isomer of 3,4-methylenedioxymethamphetamine, or said racemic mixture of 3,4-methylenedioxymethamphetamine is about 30 milligrams to about 125 milligrams.

18. The sexual therapy formulation as recited in claim 14, wherein said libido stimulation component consists of an amount of 3,4-methylenedioxymethamphetamine, said R(−) isomer of 3,4-methylenedioxymethamphetamine, said S(+) isomer of 3,4-methylenedioxymethamphetamine, or said racemic mixture of 3,4-methylenedioxymethamphetamine.

19. The sexual therapy formulation as recited in claim 18, wherein a unit dosage of said formulation comprises a therapeutically effective amount of said libido stimulation component consisting of 3,4-methylenedioxymethamphetamine, said R(−) isomer of 3,4-methylenedioxymethamphetamine, said S(+) isomer of 3,4-methylenedioxymethamphetamine, or said racemic mixture of 3,4-methylenedioxymethamphetamine.

20. The sexual therapy formulation as recited in claim 19, wherein said therapeutically effective amount of 3,4-methylenedioxymethamphetamine, said R(−) isomer of 3,4-methylenedioxymethamphetamine, said S(+) isomer of 3,4-methylenedioxymethamphetamine, or said racemic mixture of 3,4-methylenedioxymethamphetamine is about 30 milligrams to about 125 milligrams.
